(19) United States
(12) Patent Application Publication
(10) Pub. No.: US 2006/0078522 A1
(43) Pub. Date: Apr. 13, 2006

(54) Composition for treating keratin materials comprising at least one electrophilic monomer and at least one salt

(76) Inventor: Gabin Vic, Venette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/247,186

(22) Filed: Oct. 12, 2005

Related U.S. Application Data
(60) Provisional application No. 60/646,499, filed on Jan. 25, 2005.

(30) Foreign Application Priority Data
Oct. 13, 2004 (FR) 04 10811

(51) Int. Cl. A61K 8/81

(52) U.S. Cl. 424/70.11; 424/70.16

(57) ABSTRACT

Disclosed herein is a cosmetic composition for treating keratin materials, for example, keratin fibers such as the hair, comprising, in a cosmetically acceptable medium, at least one electrophilic monomer and at least one organic or mineral salt. Also disclosed is a method for cosmetically treating keratin materials comprising applying the cosmetic composition to the keratin materials.

This application claims benefit of U.S. Provisional Application No. 60/646,499, filed Jan. 25, 2005, the contents of which are incorporated herein by reference. This application also claims benefit of priority under 35 U.S.C. § 119 to French Patent Application No. 0410811, filed Oct. 13, 2004, the contents of which are also herein incorporated by reference.

Disclosed herein are compositions comprising at least one in-situ polymerizable monomer, at least one cosmetically acceptable medium, and at least one salt. Also disclosed herein are methods for cosmetically treating keratin materials, for example, keratin fibers such as the hair, comprising applying these compositions to the keratin materials.

As used herein, the term “keratin materials” means keratin fibers, for example, the hair.

Cosmetic compositions comprising silicones or polymers with high affinity for keratin materials such as the hair, are generally used to modify the surface properties of the keratin materials, for example, to condition the keratin materials and/or give them sheen. It is generally necessary to renew these treatments because conditioning agents have a tendency to be removed, especially upon shampooing.

It is theoretically possible to increase the permanence of the polymer deposit by performing a free-radical polymerization of certain monomers directly on keratin materials. However, the treatments thus obtained are cosmetically unacceptable. High degradation of the fiber is generally observed, probably associated with the polymerization initiators, and the treated hair is difficult to disentangle. As such, it is sought to obtain cosmetic compositions that can give keratin materials softness and long-lasting sheen.

Applicant has discovered that it is possible to obtain a essentially hard coating that gives keratin materials improved and long-lasting sheen by using a composition comprising at least one electrophilic monomer, such as those described in French Patent Application No. 2 840 208, at least one cosmetically acceptable medium, and at least one organic or mineral salt.

The Applicant has also discovered that, upon application of the cosmetic compositions described herein to keratin materials, a lubricating and shiny permanent coating is formed in situ.

Depending on their nature and their concentration, the at least one salt added to the composition may reinforce the rigidity of the coating and/or make it more adherent. Such a coating may provide, in addition to sheen and softness, improved permanence of the hairstyling properties.

Moreover, the Applicant has found that the keratin fibers remain perfectly individualized and can be styled without problem, and that the conditioning and sheen of the fibers have improved resistance to shampooing.

Thus, disclosed herein is a cosmetic composition for treating keratin materials, for example, keratin fibers such as the hair, comprising, in a cosmetically acceptable medium, at least one electrophilic monomer and at least one non-reducing organic or mineral salt having a melting point of greater than 60° C., with the proviso that, if the salt is an organic or mineral salt whose anion contains at least one sulfur atom, then the surface tension of this salt at a concentration of 1% by weight in water is greater than or equal to 60 mN/m.

Also disclosed herein is a method for cosmetically treating keratin materials, for example, keratin fibers such as the hair, comprising applying the cosmetic compositions described herein to the keratin materials.

Further disclosed herein is a method for imparting keratin materials with an improved and long-lasting sheen comprising applying the composition of the present disclosure to the keratin materials

Still further disclosed herein is a kit comprising a first composition comprising at least one electrophilic monomer (present, for example, in an amount ranging from 0.5% to 50% by weight relative to the total weight of the first composition) and optionally at least one anionic and/or free-radical polymerization inhibitor (present, for example, in an amount ranging from 10 ppm to 5% by weight relative to the total weight of the first composition), and a second composition comprising, in a cosmetically acceptable medium, at least one organic or mineral salt described herein (present, for example, in an amount ranging from 0.001% to 5% by weight relative to the total weight of the second composition).

Other characteristics, aspects, subjects, and advantages of the invention will become apparent on reading the description and the examples that follow.

As used herein, the term “electrophilic monomer” means a monomer capable of polymerizing via anionic polymerization in the presence of a nucleophilic agent, for instance, the hydroxyl ions (OH—) contained in water.

As used herein, the term “anionic polymerization” refers to the mechanism defined in J. March, Advanced Organic Chemistry, 3rd ed., pages 151 to 161.

Electrophilic Monomers

The at least one electrophilic monomer present in the composition disclosed herein may be chosen from:
benzylidene malonitrile derivatives (A), 2-(4-chloro-benzylidene)malononitrile (A1), ethyl 2-cyano-3-phenylacrylate (B), and ethyl 2-cyano-3-(4-chlorophenyl)acrylate (B1) described, for example, in Sayyah, J. Polymer Research, p. 97 (2000): 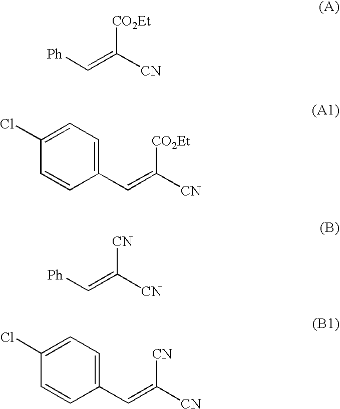
methylidenemalonate derivatives, for instance:
   diethyl 2-methylenemalonate (C) described, for example, in Hopff, Makromoleculare Chemie, p. 95 (1961), De Keyser, J. Pharm. Sci., p. 67 (1991), and Klemarczyk, Polymer, p. 173 (1998): 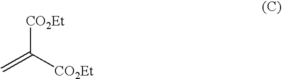
ethyl 2-ethoxycarbonylmethylenecarbonylacrylate (D) described, for example, in Breton, Biomaterials, p. 271 (1998) and Couvreur, Pharmaceutical Research, p. 1270 (1994). 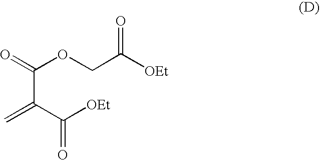
itaconate and itaconimide derivatives, for instance:
   dimethyl itaconate (E) described, for example, in Bachrach, European Polymer Journal, p. 563 (1976): 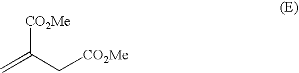
   N-butyl itaconimide (F), N-(4-tolyl) itaconimide (G), N-(2-ethylphenyl) itaconimide (H), N-(2,6-diethylphenyl) itaconimide (I) described, for example, in Wanatabe, J. Polymer Science: Part A: Polymer Chemistry, p. 2073 (1994): 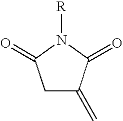
(F): R=Bu; (G): R=4-tolyl; (H): R=2-ethylphenyl; (I): R=2,5-diethylphenyl
the derivatives methyl α-(methylsulfonyl)acrylate (K), ethyl α-(methylsulfonyl)acrylate (L), methyl α-(tert-butylsulfonyl)acrylate (M), tert-butyl α-(methylsulfonyl)acrylate (N) and tert-butyl α-(tert-butylsulfonyl)acrylate (O) described, for example, in Gipstein, J. Org. Chem, p. 1486 (1980): 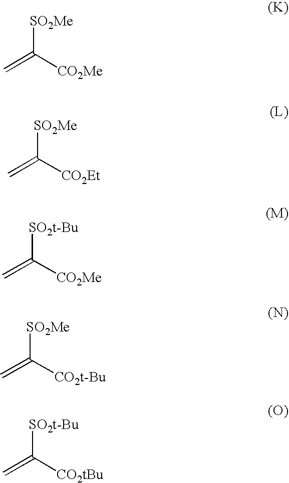
the derivatives 1,1-bis(methylsulfonyl)ethylene (P), 1-acetyl-1-methylsulfonylethylene (O), methyl α-(methylsulfonyl)vinylsulfonate (R), and α-methylsulfonylacrylonitrile (S) described, for example, in U.S. Pat. No. 2,748,050: 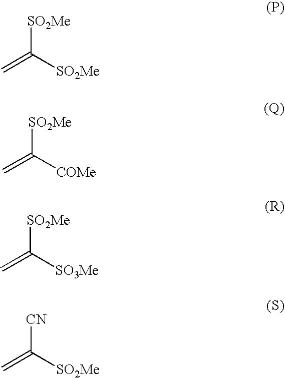
the methyl vinyl sulfone (T) and phenyl vinyl sulfone (U) derivatives described, for example, in Boor, J. Polymer Science, p. 249 (1971): 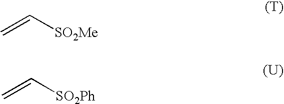
the phenyl vinyl sulfoxide derivative (V) described, for example, in Kanga, Polymer Preprints (ACS, Division of Polymer Chemistry), p. 322 (1987): 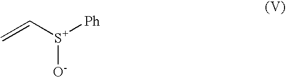
the derivative 3-methyl-N-(phenylsulfonyl)-1-aza-1,3-butadiene (W) described, for example, in Bonner, Polymer Bulletin, p. 517 (1992): 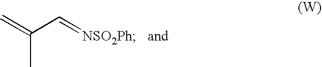
acrylate and acrylamide derivatives, for instance:
   N-propyl-N-(3-triisopropoxysilylpropyl)acrylamide (X) and N-propyl-N-(3-triethoxysilylpropyl)acrylamide (Y) described, for example, in Kobayashi, Journal of Polymer Science, Part A: Polymer Chemistry, p. 2754 (2005). 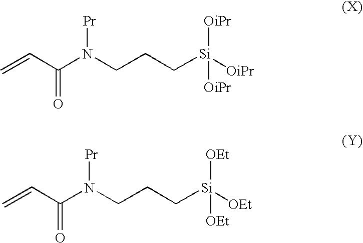
2-hydroxyethyl acrylate (Z) and 2-hydroxyethyl methacrylate (AA) described, for example, in Rozenberg, International Journal of Plastics Technology, p. 17 (2003): 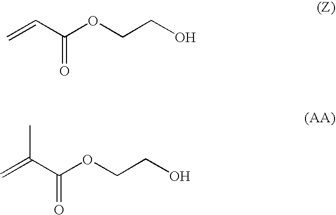
N-butyl acrylate (AB) described, for example, in Schmitt, Macromolecules, p. 2115 (2001): 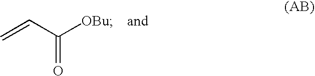
Tert-butyl acrylate (AC) described, for example, in Ishizone, Macromolecules, p. 955 (1999):

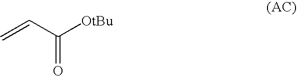

The at least one electron-withdrawing monomer may be cyclic or linear. In at least one embodiment of the present disclosure, if the monomer is cyclic, the electron-withdrawing group may be exocyclic, i.e., it does not form an integral part of the cyclic structure of the monomer.

According to one embodiment of the present disclosure, the at least one monomer may contain at least two electron-withdrawing groups. Examples of monomers comprising at least two electron-withdrawing groups include, but are not limited to, monomers of formula (I): 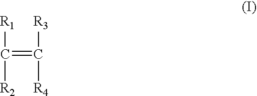
in which:
R₁ and R₂ are, independently of each other, minimally or non-electron-withdrawing groups (minimally or non-inductive-withdrawing groups) chosen from:
   hydrogen,
   saturated, unsaturated, linear, branched, and cyclic hydrocarbon-based groups comprising, for example, from 1 to 20, such as from 1 to 10 carbon atoms, and optionally comprising at least one atom chosen from nitrogen, oxygen, and sulfur, and optionally substituted with at least one group chosen from —OR, —COOR, —COR, —SH, —SR, —OH, and halogen atoms,
   modified or unmodified polyorganosiloxane residues, and
   polyoxyalkylene groups,
R₃ and R₄ are, independently of each other, electron-withdrawing (or inductive-withdrawing) groups chosen, for example, from —N(R)₃⁺, —S(R)₂⁺, —SH₂⁺, —NH₃⁺, —NO₂, —SO₂R, —C≡N, —COOH, —COOR, —COSR, —CONH₂, —F, —Cl, —Br, —I, —OR, —COR, —SH, —SR, and —OH groups, linear or branched alkenyl groups, linear or branched alkynyl groups, C₁-C₄ mono- or polyfluoroalkyl groups, aryl groups such as phenyl, and aryloxy groups such as phenoxyloxy, and
R is chosen from saturated, unsaturated, linear, branched, and cyclic hydrocarbon-based groups comprising, for example, from 1 to 20, such as from 1 to 10 carbon atoms, and optionally comprising at least one atom chosen from nitrogen, oxygen, and sulfur, and optionally substituted with at least one group chosen from —OR′, —COOR′, —COR′, —SH, —SR′, —OH, and halogen atoms, and polymer residues which may be obtained by free-radical polymerization, polycondensation, or ring opening, wherein R′ is chosen from C₁-C₁₀ alkyl radicals.

As used herein, the term “electron-withdrawing or inductive-withdrawing group (—I)” means any group that is more electronegative than carbon. Reference may be made, for example, to P. R. Wells, Prog. Phys. Org. Chem., vol. 6, page 111 (1968).

As used herein, the term “minimally or non-electron-withdrawing group” means any group whose electronegativity is less than or equal to that of carbon.

In at least one embodiment of the present disclosure, the alkenyl and/or alkynyl groups of R₃ and R₄ may comprise from 2 to 20 carbon atoms, for example, from 2 to 10 carbon atoms.

Non-limiting examples of saturated or unsaturated, linear, branched, or cyclic hydrocarbon-based groups comprising from 1 to 20 carbon atoms, or from 1 to 10 carbon atoms, include, for example linear or branched alkyl groups, linear or branched alkenyl groups, and linear or branched alkynyl groups, such as methyl, ethyl, n-butyl, tert-butyl, isobutyl, pentyl, hexyl, octyl, butenyl, and butynyl groups; cycloalkyl groups, and aromatic groups.

Examples of substituted hydrocarbon-based groups include, but are not limited to, hydroxyalkyl and polyhaloalkyl groups.

Examples of unmodified polyorganosiloxanes include, but are not limited to, polyalkylsiloxanes such as polydimethylsiloxanes, polyarylsiloxanes, such as polyphenylsiloxanes, and polyarylalkylsiloxanes, such as polymethylphenylsiloxanes.

Suitable modified polyorganosiloxanes include, but are not limited to, polydimethylsiloxanes comprising polyoxyalkylene, siloxy, silanol, amine, imine, and/or fluoroalkyl groups.

Non-limiting examples of polyoxyalkylene groups include polyoxyethylene groups and polyoxypropylene groups comprising, for example, from 1 to 200 oxyalkylene units.

Examples of mono- or polyfluoroalkyl groups include, but are not limited to, groups such as —(CH₂)ₙ—(CF₂)ₘ—CF₃ or —(CH₂)n-(CF₂)m-CHF₂, wherein n is a number ranging from 1 to 20 and m is a number ranging from 1 to 20.

In accordance with at least one embodiment of the present disclosure, the substituents R₁ to R₄ may optionally be substituted with at least one group having cosmetic activity. Cosmetic activities may be obtained, for example, from groups comprising at least one coloring, antioxidant, UV-screening, and/or conditioning function.

Groups comprising a coloring function include, but are not limited to, azo, quinone, methine, cyanomethine, and triarylmethane groups.

Examples of groups comprising an antioxidant function include, but are not limited to, butylhydroxyanisole (BHA), butylhydroxytoluene (BHT), and vitamin E groups.

Non-limiting examples of groups comprising a UV-screening function include benzophenone, cinnamate, benzoate, benzylidenecamphor, and dibenzoylmethane groups.

Suitable groups comprising a conditioning function include, for example, cationic groups and groups of fatty ester type.

In at least one embodiment of the present disclosure, the at least one electrophilic monomer may be chosen from monomers of the cyanoacrylate family and the derivatives thereof of formula (II): 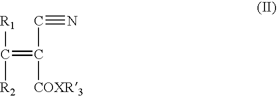
wherein,
   X is chosen from NH, S, and O,
   R₁ and R₂ are, independently of each other, minimally or non-electron-withdrawing groups (minimally or non-inductive-withdrawing groups) chosen from:
      hydrogen,
      saturated, unsaturated, linear, branched, and cyclic hydrocarbon-based groups comprising, for example, from 1 to 20, or from 1 to 10 carbon atoms, and optionally comprising at least one atom chosen from nitrogen, oxygen, and sulfur, and optionally substituted with at least one group chosen from —OR, —COOR, —COR, —SH, —SR, —OH, and halogen atoms,
      modified or unmodified polyorganosiloxane residues, and
      polyoxyalkylene groups,
   R′₃ is chosen from hydrogen and a radical R, wherein R is chosen from saturated, unsaturated, linear, branched, and cyclic hydrocarbon-based groups comprising, for example, from 1 to 20, such as from 1 to 10 carbon atoms, and optionally comprising at least one atom chosen from nitrogen, oxygen, and sulfur, and optionally substituted with at least one group chosen from —OR′, —COOR′, —COR′, —SH, —SR′, —OH, and halogen atoms, and polymer residues which may be obtained by free-radical polymerization, polycondensation, or ring opening, wherein R′ is chosen from C₁-C₁₀ alkyl radicals.

In one embodiment, X may be O.

Compounds of formula (II) may include, for example the following monomers:

a) monomers belonging to the family of C₁-C₂₀ polyfluoroalkyl 2-cyanoacrylates such as:

the ester 2,2,3,3-tetrafluoropropyl 2-cyano-2-propenoate of formula (III):

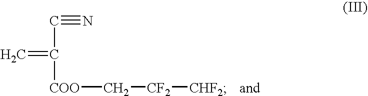

the ester 2,2,2-trifluoroethyl 2-cyano-2-propenoate of formula (IV):

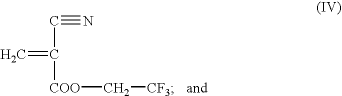

b) C₁-C₁₀ alkyl cyanoacrylates and (C₁-C₄ alkoxy)(C₁-C₁₀ alkyl) cyanoacrylates.

In one embodiment, the at least one electrophilic monomer may be chosen, for example, from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

In another embodiment, the at least one electrophilic monomer may be chosen from C₁-C₁₀ alkyl cyanoacrylates and (C₁-C₄ alkoxy)(C₁-C₁₀ alkyl) cyanoacrylates.

In a further embodiment, the at least one electrophilic monomer may be chosen from monomers of formula (V) and mixtures thereof:

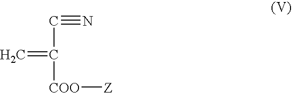

wherein Z is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃ groups.

The monomers used in accordance with the present disclosure may, in at least one embodiment, be covalently bonded to at least one support such as polymers, oligomers, and dendrimers. The polymer or oligomer may be linear, branched, in comb form, or in block form. The distribution of the monomers of the invention over the polymeric, oligomeric, or dendritic support may be chosen from random distributions, end position distributions, and blocks distributions.

According to the present disclosure, the at least one electrophilic monomer may be chosen from monomers capable of polymerizing on the keratin materials under cosmetically acceptable conditions. In at least one embodiment, the polymerization of the monomer may be performed at a temperature of less than or equal to 80° C., for example, at a temperature ranging from 10 to 80° C., or from 20 to 80° C., which does not prevent the application from being completed by a drying operation.

The at least one electrophilic monomer described herein may be present in the cosmetic composition in an amount ranging from 0.001% to 80% by weight, for example, from 0.1% to 40% by weight, or from 1% to 20% by weight relative to the total weight of the cosmetic composition.

Cosmetically Acceptable Medium

As used herein, the term “cosmetically acceptable medium” means a medium that is compatible with keratin materials.

In accordance with least one embodiment of the present disclosure, the cosmetically acceptable medium may be anhydrous. As used herein, the term “anhydrous medium” means a medium containing less than 1% by weight of water relative to the total weight of the composition.

The cosmetically acceptable medium may be chosen, for example, from organic oils; silicones such as volatile silicones, amino or non-amino silicone gums or oils, and mixtures thereof; mineral oils; plant oils such as olive oil, castor oil, rapeseed oil, coconut oil, wheatgerm oil, sweet almond oil, avocado oil, macadamia oil, apricot oil, safflower oil, candlenut oil, false flax oil, tamanu oil, and lemon oil; waxes; and organic compounds such as C₅-C₁₀ alkanes, acetone, methyl ethyl ketone, esters of C₁-C₂₀ acids and of C₁-C₈ alcohols such as methyl acetate, butyl acetate, ethyl acetate, and isopropyl myristate, dimethoxyethane, diethoxyethane, C₁₀-C₃₀ fatty alcohols such as lauryl alcohol, cetyl alcohol, stearyl alcohol, and behenyl alcohol; C₁₀-C₃₀ fatty acids such as lauric acid and stearic acid; C₁₀-C₃₀ fatty amides such as lauric diethanolamide; C₁₀-C₃₀ fatty alkyl esters such as C₁₀-C₃₀ fatty alkyl benzoates; and mixtures thereof.

In at least on embodiment of the present disclosure, the organic compounds are chosen from compounds that are liquid at a temperature of 25° C. and a pressure of 105 Pa (760 mm Hg).

Optional Additives

Polymerization inhibitors, for example, anionic and/or free-radical polymerization inhibitors, may be introduced into the compositions described herein, in order to enhance the stability of the composition over time. Non-limiting examples of polymerization inhibitors include: sulfur dioxide; nitric oxide; lactone; boron trifluoride; hydroquinone and derivatives thereof such as hydroquinone monoethyl ether; tert-butylhydroquinone (TBHQ); benzoquinone and derivatives thereof such as duroquinone; catechol and derivatives thereof such as t-butylcatechol and methoxycatechol; anisole and derivatives thereof such as methoxyanisole, hydroxyanisole, and butylhydroxyanisole; pyrogallol; 2,4-dinitrophenol; 2,4,6-trihydroxybenzene; p-methoxyphenol; hydroxybutyltoluene; alkyl sulfates; alkyl sulfites; alkyl sulfones; alkyl sulfoxides; alkyl sulfides; mercaptans; 3-sulfonene; and mixtures thereof. The alkyl groups may be chosen from groups comprising from 1 to 6 carbon atoms.

It is also possible to use organic or mineral acids, the latter containing at least one group chosen from carboxylic and sulfonic groups, with a pKa ranging from 0 to 6, such as phosphoric acid, hydrochloric acid, nitric acid, benzenesulfonic acid, toluenesulfonic acid, sulfuric acid, carbonic acid, hydrofluoric acid, acetic acid, formic acid, propionic acid, benzoic acid, mono-, di- or trichloroacetic acid, salicylic acid, and trifluoroacetic acid.

The inhibitor may be present in the composition in an amount ranging from 10 ppm to 20%, for example, from 10 ppm to 5%, or from 10 ppm to 1% by weight relative to the total weight of the composition.

The compositions in accordance with the disclosure may also contain at least one agent conventionally used in cosmetics, for instance, reducing agents, fatty substances, plasticizers, softeners, antifoams, moisturizers, pigments, clays, mineral fillers, UV-screening agents, mineral colloids, peptizers, solubilizing agents, fragrances, preserving agents, anionic surfactants, cationic surfactants, nonionic surfactants, amphoteric surfactants, fixing polymers, non-fixing polymers, polyols, proteins, vitamins, direct dyes, oxidation dyes, nacreous agents, propellants, mineral or organic thickeners such as benzylidene sorbitol, and N-acylamino acids. These agents may optionally be encapsulated, for example, in a capsule of polycyanoacrylate type.

Organic or Mineral Salt

As used herein, the term “organic or mineral salt” means any non-polymeric compound formed by the reaction of an organic or mineral acid with an organic or mineral base.

According to at least one embodiment, the salts of the present disclosure may be soluble in the medium containing the at least one electrophilic monomer. As used herein, the term “soluble” means the formation of a macroscopically homogeneous solution.

If the salt is derived from an organic base, its surface tension at 1% in water may be greater than 60 mN/m.

Non-limiting examples of suitable mineral salts include sodium chloride, magnesium chloride, and copper sulfate.

Examples of organic salts include, but are not limited to, salts formed by reacting at least one anion chosen from phosphates, borates, silicates, bicarbonates, carbonates, chlorates, nitrates, silicates, sulfonate, comprising at least one saturated or unsaturated, cyclic or non-cyclic C₁-C₃₀ hydrocarbon-based chain, optionally substituted with at least one group chosen from hydroxyl, C₁-C₄ alkoxy, and halogen groups, with at least one cation chosen from potassium, sodium, strontium, cadmium, calcium, ammonium (such as tetraalkylammonium and arylammonium), phosphonium, barium, lithium, and magnesium.

Suitable organic salts include, but are not limited to, sodium monobutyl and dibutyl phosphates and sodium monoethyl and diethyl phosphates.

The at least one organic or mineral salt may be present in the composition in an amount ranging from 0.001% to 70% by weight, for example, from 0.01% to 20%, or from 0.1% to 10% by weight relative to the total weight of the composition.

Cosmetic Treatment Method

Disclosed herein is a method for cosmetically treating keratin materials comprising applying the cosmetic composition described herein to the keratin materials. The application optionally may be carried out in the presence of at least one nucleophilic agent with or without heating.

The nucleophilic agent may be, for example, water. Water may be provided by wetting the keratin materials before application of the cosmetic composition.

It is also possible, in order to modify the reaction kinetics, to wet the keratin materials before application using an aqueous solution whose pH has been adjusted using a base, an acid, or an acid/base mixture. The acid and/or base may be mineral or organic.

These two operations may also be performed after applying the composition.

The anionic polymerization kinetics may also be modified by preimpregnating the keratin materials with at least one nucleophilic agent other than water. The nucleophilic agent may be pure, in the form of a solution, in the form of an emulsion, or may be encapsulated.

Nucleophilic agents capable of initiating the anionic polymerization are systems known in the art, which are capable of generating a carbanion upon contact with a nucleophilic agent, such as the hydroxyl ions contained in water. As used herein, the term “carbanion” refers to the chemical species defined in J. March, Advanced Organic Chemistry, 3rd ed., page 141.

The nucleophilic agents may comprise at least one entity chosen from molecular compounds, oligomers, dendrimers, and polymers comprising nucleophilic functions. Examples of nucleophilic functions include, but are not limited to, R₂N⁻, NH₂⁻, Ph₃C⁻, R₃C⁻, PhNH⁻, pyridine, ArS⁻, R—C≡C⁻, RS⁻, SH, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, and H₂O, wherein Ph represents a phenyl group; Ar represents an aryl group, and R represents a C₁-C₁₀ alkyl group.

To modify the anionic polymerization kinetics, the nucleophilicity of the keratin material may be increased via chemical conversion of the keratin material. Examples that may be mentioned include the reduction of the disulfide bridges, of which keratin is partly composed, into thiols, before applying the composition of the present disclosure. Examples of reducing agents for the disulfide bridges include, but are not limited to:
anhydrous sodium thiosulfate,
powdered sodium metabisulfite,
thiourea,
ammonium sulfite,
thioglycolic acid,
thiolactic acid,
ammonium thiolactate,
glyceryl monothioglycolate,
ammonium thioglycolate,
thioglycerol,
2,5-dihydroxybenzoic acid,
diammonium dithioglycolate,
strontium thioglycolate,
calcium thioglycolate,
zinc formosulfoxylate,
isooctyl thioglycolate,
dl-cysteine, and
monoethanolamine thioglycolate.

The viscosity of the composition of the present disclosure may also be increased so as to modify the anionic polymerization kinetics, for example, to reduce the rate of polymerization of the monomers of the present disclosure. The viscosity of the composition may be increased by adding to the composition at least one polymer having no reactivity towards the monomers. Suitable non-reactive polymers include, but are not limited to poly(methyl methacrylate) (PMMA) copolymers and cyanoacrylate-based copolymers, described, for example, in U.S. Pat. No. 6,224,622.

In order to improve, inter alia, the adhesion of the poly(cyanoacrylate) formed in situ, the keratin materials may be pretreated with polymers of any type. Optionally, a hair treatment may be performed before applying the composition of the present disclosure to the keratin materials, for instance, a direct dyeing or oxidation dyeing, permanent-waving, and/or hair relaxing operation.

The application of the compositions may or may not be followed by rinsing. The compositions of the present disclosure may be in various forms, such as lotions, sprays, and mousses, and may be applied in the form of a shampoo or a hair conditioner.

The hair treatment method may include (a) applying at least one salt as defined above to the keratin materials and (b) applying at least one electrophilic monomer to the keratin materials, the order of the steps being irrelevant. In one embodiment, the application of the at least one salt is performed before the application of the at least one electrophilic monomer.

Also disclosed herein is a method for imparting keratin materials with an improved and long-lasting sheen comprising applying the composition of the present disclosure to the keratin materials.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The percentages are expressed on a weight basis of active material, unless otherwise indicated.

### EXAMPLES

### Example 1

A sheen cream in accordance with the present disclosure (sheen cream 2) and a comparative sheen cream (sheen cream 1) were prepared by combining the following components:

Sheen Cream 1 (without Salt)

| | |
|---|---|
| n-octyl 2-cyanoacrylate¹ | 5% |
| Cyclopentasiloxane | 47.4% |
| Cyclopentasiloxane dimethicone copolyol | 47.4% |
| Fragrance | 0.2% |

¹Rite Lok CON895, sold by Chemence

Sheen Cream 2 (with Salt)

| | |
|---|---|
| n-octyl 2-cyanoacrylate¹ | 5% |
| Cyclopentasiloxane | 46.65% |
| cyclopentasiloxane dimethicone copolyol | 46.65% |
| Sodium diethyl phosphate | 1.5% |
| Fragrance | 0.2% |

¹Rite Lok CON895, sold by Chemence

Two locks of sensitized hair (2.7 g) were moistened with 1 ml of water per lock. Two grams of the compositions described above were applied to the moistened locks. After application, the locks of hair were dried under a drying hood for 30 minutes at 40° C.

For each lock, the feel and the sheen of the hair were evaluated by a panel of 10 people. An untreated lock of the same nature was used as reference. The tactile and visual evaluation of the various locks of hair was repeated with the same procedure after washing 5 times in succession with a shampoo sold under the name DOP camomile.

| | Nature of the | | Sheen cream 2 |
|---|---|---|---|
| | treatment | Sheen cream 1 | of the invention |
| Sensory | After application | Softness 5 | Softness 5 |
| evaluation* | | Sheen 5 | Sheen 5 |
| | | Tack 1 | Tack 5 |
| | After 5 shampoo | Softness 4 | Softness 4 |
| | washes | Sheen 4 | Sheen 4 |
| | | Tack 0 | Tack 4 |

*Grading:
0 = equivalent to the untreated lock
5 = very much greater than the untreated lock

The experiment shows that the cosmetic properties (softness, sheen and tack) provided by the composition of the invention are improved as compared to the cosmetic properties provided by cream 1, immediately after applying the compositions. Furthermore, after performing 5 shampoo washes, the softness, sheen, and tack provided by the composition of the invention are conserved.

The composition of the invention makes it possible, after several shampoo washes, to maintain the softness, sheen, and tack of the hair, without reapplication of the composition.

It should be noted that the levels of sheen and softness are higher with the composition of the invention when it is applied to moistened hair (application mode No. 2).

### Example 2

Sheen Cream 3

| | |
|---|---|
| methylheptyl cyanoacrylate¹ | 5% |
| Cyclopentasiloxane | 46.65% |
| cyclopentasiloxane dimethicone copolyol | 46.65% |
| Sodium dicetyl phosphate | 1.5% |
| Fragrance | 0.2% |

¹sold by Chemence

### Example 3

Sheen Cream 4

| | |
|---|---|
| ethoxyethyl cyanoacrylate¹ | 5% |
| Cyclopentasiloxane | 45.65% |
| cyclopentasiloxane dimethicone copolyol | 46.65% |
| Sodium dicetyl phosphate | 1.5% |
| acetic acid | 1% |
| Fragrance | 0.2% |

¹EO 460 sold by Tong Shen

### Example 4

Sheen Cream 5

| | |
|---|---|
| butyl cyanoacrylate¹ | 5% |
| Cyclopentasiloxane | 45.65% |
| cyclopentasiloxane dimethicone copolyol | 46.65% |
| acetic acid | 1% |
| Sodium dicetyl phosphate | 1.5% |
| Fragrance | 0.2% |

¹B 60 sold by Tong Shen

### Example 5

Sheen Cream 6

| | |
|---|---|
| ethylhexyl cyanoacrylate¹ | 5% |
| Cyclopentasiloxane | 46.65% |
| cyclopentasiloxane dimethicone copolyol | 46.65% |
| Sodium dicetyl phosphate | 1.5% |
| Fragrance | 0.2% |

¹O-60 sold by Tong Shen

### Example 6

Sheen Cream 7

| | |
|---|---|
| methylheptyl cyanoacrylate¹ | 4.5% |
| ethylhexyl cyanoacrylate² | 0.5% |
| Cyclopentasiloxane | 46.65% |
| cyclopentasiloxane dimethicone copolyol | 46.65% |
| Sodium dicetyl phosphate | 1.5% |
| Fragrance | 0.2% |

¹sold by Chemence
²O-60 sold by Tong Shen

### Example 7

Sheen Cream 8

| | |
|---|---|
| methylheptyl cyanoacrylate¹ | 3.5% |
| butyl cyanoacrylate² | 1.5% |
| Cyclopentasiloxane | 46.65% |
| cyclopentasiloxane dimethicone copolyol | 46.65% |
| sodium dicetyl phosphate | 1.5% |
| Fragrance | 0.2% |

¹sold by Chemence
²B-60 sold by Tong Shen

What is claimed is:

1. A cosmetic composition comprising, in a cosmetically acceptable medium, at least one electrophilic monomer and at least one non-reducing organic or mineral salt having a melting point of greater than 60° C., with the proviso that if the salt is an organic salt whose anion contains at least one sulfur atom, then the surface tension of this salt at a concentration of 1% by weight in water is greater than or equal to 60 mN/m.

2. The composition of claim 1, wherein the at least one electrophilic monomer is chosen from monomers of formula (I): 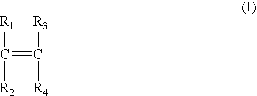
wherein,
R₁ and R₂ are, independently of each other, minimally or non-electron-withdrawing groups chosen from:
hydrogen,
saturated, unsaturated, linear, branched, and cyclic hydrocarbon-based groups comprising from 1 to 20 carbon atoms, and optionally comprising at least one atom chosen from nitrogen, oxygen, and sulfur, and optionally substituted with at least one group chosen from —OR, —COOR, —COR, —SH, —SR, —OH, and halogen atoms,
modified or unmodified polyorganosiloxane residues, and
polyoxyalkylene groups,
R₃ and R₄ are, independently of each other, electron-withdrawing groups chosen from —N(R)₃⁺, —S(R)₂⁺, —SH₂⁺, —NH₃⁺, —NO₂, —SO₂R, —C≡N, —COOH, —COOR, —COSR, —CONH₂, —F, —Cl, —Br, —I, —OR, —COR, —SH, —SR, and —OH groups, linear and branched alkenyl groups, linear and branched alkynyl groups, C₁-C₄ mono- and polyfluoroalkyl groups, aryl groups, and aryloxy groups,
R is chosen from saturated, unsaturated, linear, branched, and cyclic hydrocarbon-based groups comprising from 1 to 20 carbon atoms, optionally comprising at least one atom chosen from nitrogen, oxygen, and sulfur, and optionally substituted with at least one group chosen from —OR′, —COOR′, —COR′, —SH, —SR′, —OH, and halogen atoms, and polymer residues obtained by free-radical polymerization, polycondensation, or ring opening, wherein R′ is chosen from C₁-C₁₀ alkyl radicals.

3. The composition of claim 2, wherein the at least one electrophilic monomer is chosen from monomes of formula (II): 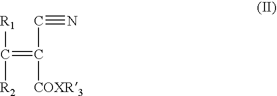
wherein,
X is chosen from NH, S, and O,
R′₃ is chosen from hydrogen and the radical R.

4. The composition of claim 3, wherein the at least one electrophilic monomer is chosen from C₁-C₂₀ polyfluoroalkyl 2-cyanoacrylates, (C₁-C₁₀) alkyl cyanoacrylates, and (C₁-C₄ alkoxy)(C₁-C₁₀ alkyl) cyanoacrylates.

5. The composition of claim 4, wherein the at least one electrophilic monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, 2-methoxyethyl 2-cyanoacrylate, 2-octyl 2-cyanoacrylate, 2-propoxyethyl 2-cyanoacrylate, n-octyl 2-cyanoacrylate, and isoamyl cyanoacrylate.

6. The composition of claim 3, wherein the at least one electrophilic monomer is chosen from monomers of formula (III): 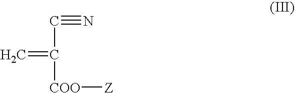
wherein, Z is chosen from —(CH₂)₇—CH₃, —CH(CH₃)—(CH₂)₅—CH₃, —CH₂—CH(C₂H₅)—(CH₂)₃—CH₃, —(CH₂)₅—CH(CH₃)—CH₃, and —(CH₂)₄—CH(C₂H₅)—CH₃.

7. The composition of claim 1, wherein the at least one electrophilic monomer is present in the composition in an amount ranging from 0.001% to 80% by weight relative to the total weight of the composition.

8. The composition of claim 7, wherein the at least one electrophilic monomer is present in the composition in an amount ranging from 0.1% to 40% by weight relative to the total weight of the composition.

9. The composition of claim 8, wherein the at least one electrophilic monomer is present in the composition in an amount ranging from 1% to 20% by weight relative to the total weight of the composition.

10. The composition of claim 1, wherein the at least one electrophilic monomer is covalently bonded to at least one support chosen from polymers, oligomers, and dendrimers.

11. The composition of claim 1, wherein the cosmetically acceptable medium is anhydrous.

12. The composition of claim 11, wherein the cosmetically acceptable medium is chosen from organic oils, silicones, mineral oils, plant oils, waxes, C₅-C₁₀ alkanes, acetone, methyl ethyl ketone, esters of C₁-C₂₀ acids, esters of C₁-C₈ alcohols, dimethoxyethane, diethoxyethane, C₁₀-C₃₀ fatty alcohols, C₁₀-C₃₀ fatty acids, C₁₀-C₃₀ fatty amides, C₁₀-C₃₀ fatty alkyl esters, and mixtures thereof.

13. The composition of claim 1, further comprising at least one polymerization inhibitor.

14. The composition of claim 13, wherein the at least one polymerization inhibitor is chosen from anionic and/or free-radical polymerization inhibitors.

15. The composition of claim 13, wherein the at least one polymerization inhibitor is chosen from sulfur dioxide; nitric oxide; lactone; boron trifluoride; hydroquinone and derivatives thereof such as hydroquinone monoethyl ether; tert-butylhydroquinone (TBHQ); benzoquinone and derivatives thereof such as duroquinone; catechol and derivatives thereof such as t-butylcatechol and methoxycatechol; anisole and derivatives thereof such as methoxyanisole, hdyroxyanisole, and butylhydroxyanisole; pyrogallol; 2,4 dinitrophenol; 2,4,6-trihydrobenzene; p-methoxyphenol; hydroxybutyltoluene; alkyl suflates; alkyl sulfites; alkyl sulfones; alkyl sulfoxides; alkyl sulfides; mercaptans; 3-sulfonene; and mixtures thereof.

16. The composition of claim 13, wherein the at least one polymerization inhibitor is present in an amount ranging from 10 ppm to 20% by weight relative to the total weight of the composition.

17. The composition of claim 16, wherein the at least one polymerization inhibitor is present in an amount ranging from 10 ppm to 5% by weight relative to the total weight of the composition.

18. The composition of claim 17, wherein the at least one polymerization inhibitor is present in an amount ranging from 10 ppm to 1% by weight relative to the total weight of the composition.

19. The composition of claim 1, wherein the at least one salt is a mineral salt chosen from sodium chloride, magnesium chloride, and copper sulfate.

20. The composition of claim 1, wherein the at least one salt is chosen from organic salts formed by reacting at least one anion chosen from phosphates, borates, silicates, bicarbonates, carbonates, chlorates, nitrates, silicates, and sulfonate, comprising at least one saturated or unsaturated, cyclic or non-cyclic C₁-C₃₀ hydrocarbon-based chain, optionally substituted with at least one group chosen from hydroxyl, C₁-C₄ alkoxy, and halogen groups, with at least one cation chosen from potassium, sodium, strontium, cadmium, calcium, ammonium, phosphonium, barium, lithium, and magnesium.

21. The composition of claim 20, wherein the organic salt is chosen from sodium monobutyl phosphates, sodium dibutyl phosphates, sodium monoethyl phosphates, and sodium diethyl phosphates.

22. The composition of claim 1, wherein the at least one organic or mineral salt is present in the composition in an amount ranging from 0.001% to 70% by weight relative to the total weight of the composition.

23. The composition of claim 1, wherein the at least one organic or mineral salt is present in the composition in an amount ranging from 0.01% to 20% by weight relative to the total weight of the composition.

24. The composition of claim 1, wherein the at least one organic or mineral salt is present in the composition in an amount ranging from 0.1% to 10% by weight relative to the total weight of the composition.

25. The composition of claim 1, further comprising at least one agent chosen from reducing agents, fatty substances, plasticizers, softeners, antifoams, moisturizers, pigments, clays, mineral fillers, UV-screening agents, mineral colloids, peptizers, solubilizing agents, fragrances, preserving agents, anionic surfactants, cationic surfactants, nonionic surfactants, amphoteric surfactants, fixing polymers, non-fixing polymers, polyols, proteins, vitamins, direct dyes, oxidation dyes, nacreous agents, propellants, mineral or organic thickeners, and N-acylamino acids.

26. The composition of claim 25, wherein the at least one agent is encapsulated.

27. The composition of claim 1, wherein the composition is in the form chosen from lotions, sprays, and mousses.

28. A method for cosmetically treating keratin materials comprising applying, in any order, at least one non-reducing organic or mineral salt having a melting point of greater than 60° C. and at least one electrophilic monomer to the keratin materials, with the proviso that if the salt is an organic salt whose anion contains at least one sulfur atom, then the surface tension of this salt at a concentration of 1% by weight in water is greater than or equal to 60 mN/m.

29. The method of claim 28, wherein the at least one salt is applied before the at least one electrophilic monomer.

30. A method for cosmetically treating keratin materials comprising applying a cosmetic composition to the keratin materials, in the presence of at least one nucleophilic agent,
wherein the cosmetic composition comprises, in a cosmetically acceptable medium, at least one electrophilic monomer and at least one non-reducing organic or mineral salt having a melting point of greater than 60° C.,
with the proviso that if the salt is an organic salt whose anion contains at least one sulfur atom, then the surface tension of this salt at a concentration of 1% by weight in water is greater than or equal to 60 mN/m.

31. The method of claim 30, wherein the at least one nucleophilic agent is chosen from molecular compounds, oligomers, dendrimers, and polymers containing at least one nucleophilic function chosen from R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO3⁻, ClO₄⁻, and H₂O, wherein Ph is a phenyl group, Ar is an aryl group, and R is an C₁-C₁₀ aryl group.

32. The method of claim 30, wherein the at least one nucleophilic agent is water.

33. The method of claim 30, wherein composition is applied after moistening the keratin materials with an aqueous solution whose pH has been adjusted using a base, an acid, or an acid/base mixture.

34. The method of claim 30, wherein the keratin materials are preimpregnated using a nucleophilic agent other than water.

35. The method of claim 30, wherein the keratin materials are reduced before applying the composition.

36. The method of claim 30, wherein the keratin materials are reduced by applying at least one reducing agent to the keratin materials.

37. The method of claim 36, wherein the at least one reducing agent is chosen from anhydrous sodium thiosulfate, powdered sodium metabisulfite, thiourea, ammonium sulfite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulfoxylate, isooctyl thioglycolate, dl-cysteine, and monoethanolamine thioglycolate.

38. The method of claim 30, wherein the composition further comprises at least one polymer chosen from poly(methyl methacrylate) and cyanoacrylate-based copolymers.

39. The method of claims 30, wherein the application of the composition is followed by rinsing.

40. The method of claim 30, wherein the keratin materials are keratin fibers such as the hair.

41. A method for imparting keratin materials with improved and long-lasting sheen of the hair comprising applying a cosmetic composition to the keratin materials,
wherein the cosmetic composition comprises, in a cosmetically acceptable medium, at least one electrophilic monomer and at least one non-reducing organic or mineral salt having a melting point of greater than 60° C.,
with the proviso that if the salt is an organic salt whose anion contains at least one sulfur atom, then the surface tension of this salt at a concentration of 1% by weight in water is greater than or equal to 60 mN/m.

42. A kit comprising a first composition comprising (a) at least one electrophilic monomer and optionally at least one anionic and/or free-radical polymerization inhibitor, and (b) a second composition comprising, in a cosmetically acceptable medium, at least one non-reducing organic or mineral salt with a melting point of greater than 60° C., with the proviso that if the salt is an organic salt whose anion contains at least one sulfur atom, then the surface tension of this salt at a concentration of 1% by weight in water is greater than or equal to 60 mN/m.
